(19) United States
(12) Patent Application Publication
(10) Pub. No.: US 2024/0335169 A1
(43) Pub. Date: Oct. 10, 2024

(54) METHODS AND SYSTEMS FOR DISPLAYING QUALITY PROFILES POINTS IN AN ELECTRO-ANATOMICAL MAP

(71) Applicant: Biosense Webster (Israel) Ltd., Yokneam (IL)

(72) Inventor: Ben Ami Novogrodsky, Haifa (IL)

(73) Assignee: Biosense Webster (Israel) Ltd., Yokneam (IL)

(21) Appl. No.: 18/129,200

(22) Filed: Mar. 31, 2023

(51) Int. Cl. A61B 5/00; A61B 5/339; A61B 5/367

(52) U.S. Cl. CPC A61B 5/7221; A61B 5/339; A61B 5/367

(57) ABSTRACT

A system, method and user interface that allows a user to define a quality profile for acquiring points. The system, method and user interface divide the parameters across the quality index into groups. Such groups may include annotation, location and morphology, for example. The groups allow a user to select the weight of each group for computing the quality index. Each parameter may be assigned a score, such as from 1-5, for example, where a lower score defines a lower quality and a higher score defines a higher quality. Through a proper acquisition method, the user may be able to acquire points with a customized quality profile.

### FIELD OF INVENTION

The present invention is related to assessing the quality and acquiring points in a mapping system. More particularly, the present invention relates to quality profile of a point for quality assessment and selective point acquisition in CARTO.

### BACKGROUND

Currently, maps are taken of organs, such as the heart. These maps are composed of a series of points where data is collected. The map of an organ is based on the quality of the points that make up that mapping (also referred to as an electro-anatomical map in the case of electrophysiological mapping of the heart). The points used, or considered for use, in the mapping may be rated according to their quality level, like a quality index, which may be referred to as a SMART Index. A SMARTMAP algorithm permits users of the mapping system to customize the quality of the map by setting a threshold quality score for points including in the map, and to replace points in the mapping with higher quality points acquired by the mapping system. The quality score of a point is a weighted average of serval parameters. The score may be computed in real-time while acquisition of the points occurs. However, there are presently no techniques that provide feedback to users of the system to determine the quality of the point, or alternatively, why the point is determined to be lower quality. The quality index may include many parameters. Different points can have the same value for a quality index, but for different reasons. The user may not know which of the parameters was a dominant parameter that led to the value in the quality index.

### SUMMARY

A system, method and user interface that allows a user to define a quality profile for acquiring points. The system, method and user interface divide the parameters across the quality index into groups. Such groups may include annotation, location and morphology, for example. The groups allow a user to select the weight of each group for computing the quality index. Each parameter may be assigned a score, such as from 1-5, for example, where a lower score defines a lower quality and a higher score defines a higher quality. Through a proper acquisition method, the user may be able to acquire points with a customized quality profile.

A system, device and method for registering a mapped data point is described. The system, device and method may include a catheter, a processor and memory. The system, device and method include providing a plurality of values in a quality profile of a mapped data point of an electro-anatomical map; and displaying, via an interface, a simplex having vertices defined by each of the plurality of values. The system, device and method may include acquiring a data point if each of the plurality of values are within the defined range for a mapped data point the data point. The simplex may be a triangle and the plurality of values are composed of a first value, a second value, and a third value. The first value may include morphology quality, the second value may include annotation quality, and the third value may include location quality.

The simplex may be a tetrahedron and the plurality of values are composed of a first value, a second value, a third value, and a fourth value. The plurality of values may include underlying parameters for measured values. The one of the plurality of values that includes underlying parameters for measured values may combine the underlying parameters to determine the one value using weighting and the weighting may be equal between parameters or the weighting may include a dominant parameter(s) and other complementary parameters. The plurality of values comprises a value for pattern matching of the data point. The plurality of values comprises a value for LAT stability of the data point and a value for sharpness of the data point, and the plurality of values comprises a value for positional stability, a TPI value, a CPM value and a value for respiration. The system, device and method may further include displaying, via the interface, a visual depiction of a defined range on the simplex, the defined range including of acceptable values for the plurality of values in the quality profile.

### BRIEF DESCRIPTION OF THE DRAWINGS

A more detailed understanding may be had from the following description, given by way of example in conjunction with the accompanying drawings, wherein like reference numerals in the figures indicate like elements, and wherein:

FIG. 1 depicts an example catheter-based electrophysiology mapping and ablation system according to one or more embodiments;

FIG. 2 is a block diagram of an example system for remotely monitoring and communicating patient biometrics according to one or more embodiments;

FIG. 3 is a system diagram of an example of a computing environment in communication with network according to one or more embodiments;

FIG. 4 shows an example of a linear catheter including multiple electrodes that may be used to map a cardiac area;

FIG. 5 shows an example of a balloon catheter including multiple splines (e.g., 12 splines in the specific example of FIG. 5) including splines and multiple electrodes on each spline including electrodes;

FIG. 6 shows an example of a loop catheter (also referred to as a lasso catheter) including multiple electrodes that may be used to map a cardiac area;

FIG. 7 illustrates a quality profile of a CARTO point;

FIG. 8 illustrates a screenshot related to display including a quality profile;

FIG. 9 illustrates two example quality profiles 9A, 9B illustrating the use of the quality profile in real-time to accumulate data;

FIG. 10 illustrates a point list which may be created by acquiring points in a mapping; and

FIG. 11 illustrates a method of using the quality profile.

### DETAILED DESCRIPTION

A system, method and user interface that allows a user to define a quality profile for acquiring points. The system, method and user interface divide the parameters across the quality index into groups. Such groups may include annotation, location and morphology, for example. The groups allow a user to select the weight of each group for computing the quality index. Each parameter may be assigned a score, such as from 1-5, for example, where a lower score defines a lower quality and a higher score defines a higher quality. Through a proper acquisition method, the user may be able to acquire points with a customized quality profile.

One or more advantages, technical effects, and/or benefits can include providing cardiac physicians and medical personnel information to enhance the collection of points within an organ mapping, such as a cardiac mapping. Thus, the system, method and user interface particularly utilizes and transforms medical device equipment to enable/implement organ mapping with increased efficiency and quality that are otherwise not currently available or currently performed by cardiac physicians and medical personnel.

A system, device and method for registering a mapped data point is described. The system, device and method may include a catheter, a processor and memory. The system, device and method include providing a plurality of values in a quality profile of a mapped data point of an electro-anatomical map, and displaying, via an interface, a simplex having vertices defined by each of the plurality of values. The system, device and method may include acquiring a data point if each of the plurality of values are within the defined range for a mapped data point. The simplex may be a triangle and the plurality of values are composed of a first value, a second value, and a third value. The first value may include morphology quality, the second value may include annotation quality, and the third value may include location quality. The simplex may be a tetrahedron and the plurality of values are composed of a first value, a second value, a third value, and a fourth value. The plurality of values may include underlying parameters for measured values. The one of the plurality of values that includes underlying parameters for measured values may combine the underlying parameters to determine the one value using weighting and the weighting may be equal between parameters or the weighting may include a dominant parameter(s) and other complementary parameters. The plurality of values comprises a value for pattern matching of the data point. The plurality of values comprises a value for LAT stability of the data point and a value for sharpness of the data point, and the plurality of values comprises a value for positional stability, a TPI value, a CPM value and a value for respiration. The system, device and method may further include displaying, via the interface, a visual depiction of a defined range on the simplex, the defined range including of acceptable values for the plurality of values in the quality profile.

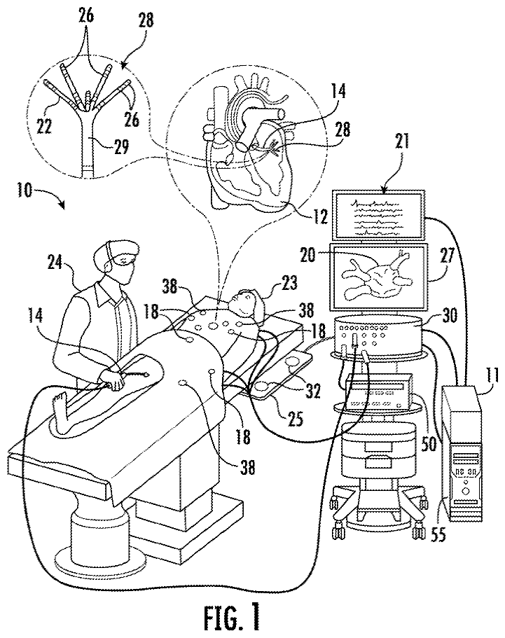

Reference is made to FIG. 1 showing an example system (e.g., medical device equipment and/or catheter-based electrophysiology mapping and ablation), shown as system 10, in which one or more features of the subject matter herein can be implemented according to one or more embodiments. All or part of the system 100 can be used to collect information (e.g., biometric data and/or a training dataset) and/or used to implement a machine learning and/or an artificial intelligence algorithm as described herein. The system 10, as illustrated, includes a recorder 11, a heart 12, a catheter 14, a model or anatomical map 20, an electrogram 21, a spline 22, a patient 23, a physician 24 (or a medical professional or clinician), a location pad 25, an electrode 26, a display device 27, a distal tip 28, a sensor 29, a coil 32, a patient interface unit (PIU) 30, electrode skin patches 38, an ablation energy generator 50, and a workstation 55. Note further each element and/or item of the system 10 is representative of one or more of that element and/or that item. The example of the system 10 shown in FIG. 1 can be modified to implement the embodiments disclosed herein. The disclosed embodiments can similarly be applied using other system components and settings. Additionally, the system 10 can include additional components, such as elements for sensing electrical activity, wired or wireless connectors, processing and display devices, or the like. While the heart 12, patient 23, and physician 24 are described in association with the example system for completeness, one of skill in the art would understand that the heart 12, patient 23, and physician 24 are not a part of the system itself, and instead the system works on or on behalf of these element.

The system 10 includes multiple catheters 14, which are percutaneously inserted by the physician 24 through the patient's vascular system into a chamber or vascular structure of the heart 12. Typically, a delivery sheath catheter is inserted into the left or right atrium near a desired location in the heart 12. Thereafter, a plurality of catheters can be inserted into the delivery sheath catheter so as to arrive at the desired location. The plurality of catheters 14 may include catheters dedicated for sensing Intracardiac Electrogram (IEGM) signals, catheters dedicated for ablating and/or catheters dedicated for both sensing and ablating. The example catheter 14 that is configured for sensing IEGM is illustrated herein. The physician 24 brings the distal tip 28 of the catheter 14 into contact with the heart wall for sensing a target site in the heart 12. For ablation, the physician 24 would similarly bring a distal end of an ablation catheter to a target site for ablating.

The catheter 14 is an exemplary catheter that includes one and preferably multiple electrodes 26 optionally distributed over a plurality of splines 22 at the distal tip 28 and configured to sense the IEGM signals. The catheter 14 may additionally include the sensor 29 embedded in or near the distal tip 28 for tracking position and orientation of the distal tip 28. Optionally and preferably, position sensor 29 is a magnetic based position sensor including three magnetic coils for sensing three-dimensional (3D) position and orientation.

The sensor 29 (e.g., a position or a magnetic based position sensor) may be operated together with the location pad 25 including a plurality of magnetic coils 32 configured to generate magnetic fields in a predefined working volume. Real time position of the distal tip 28 of the catheter 14 may be tracked based on magnetic fields generated with the location pad 25 and sensed by the sensor 29. Details of the magnetic based position sensing technology are described in U.S. Pat. Nos. 5,5391,199; 5,443,489; 5,558,091; 6,172,499; 6,239,724; 6,332,089; 6,484,118; 6,618,612; 6,690,963; 6,788,967; 6,892,091.

The system 10 includes one or more electrode patches 38 positioned for skin contact on the patient 23 to establish location reference for the location pad 25 as well as impedance-based tracking of the electrodes 26. For impedance-based tracking, electrical current is directed toward the electrodes 26 and sensed at the patches 38 (e.g., electrode skin patches) so that the location of each electrode can be triangulated via the patches 38. Details of the impedance-based location tracking technology are described in U.S. Pat. Nos. 7,536,218; 7,756,576; 7,848,787; 7,869,865; and 8,456,182, which are incorporated herein by reference.

The recorder 11 displays the electrograms 21 captured with the electrodes 18 (e.g., body surface electrocardiogram (ECG) electrodes) and intracardiac electrograms (IEGM) captured with the electrodes 26 of the catheter 14. The recorder 11 may include pacing capability for pacing the heart rhythm and/or may be electrically connected to a standalone pacer.

The system 10 may include the ablation energy generator 50 that is adapted to conduct ablative energy to the one or more of electrodes 26 at the distal tip 28 of the catheter 14 configured for ablating. Energy produced by the ablation energy generator 50 may include, but is not limited to, radiofrequency (RF) energy or pulsed-field ablation (PFA) energy, including monopolar or bipolar high-voltage DC pulses as may be used to effect irreversible electroporation (IRE), or combinations thereof.

The PIU 30 is an interface configured to establish electrical communication between catheters, electrophysiological equipment, power supply and the workstation 55 for controlling operation of the system 10. Electrophysiological equipment of the system 10 may include for example, multiple catheters 14, the location pad 25, the body surface ECG electrodes 18, the electrode patches 38, the ablation energy generator 50, and the recorder 11. Optionally and preferably, the PIU 30 additionally includes processing capability for implementing real-time computations of location of the catheters and for performing ECG calculations.

The workstation 55 includes memory, a processor unit with memory or storage with appropriate operating software loaded therein, and user interface capability. The workstation 55 may provide multiple functions, optionally including: (1) modeling the endocardial anatomy in three-dimensions (3D) and rendering the model or anatomical map 20 for display on the display device 27, (2) displaying on the display device 27 activation sequences (or other data) compiled from recorded electrograms 21 in representative visual indicia or imagery superimposed on the rendered anatomical map 20, (3) displaying real-time location and orientation of multiple catheters within the heart chamber, and (5) displaying on the display device 27 sites of interest such as places where ablation energy has been applied. One commercial product embodying elements of the system 10 is available as the CARTO™ 3 System, available from Biosense Webster, Inc., 31A Technology Drive, Irvine, CA 92618.

For instance, the system 10 can be part of a surgical system (e.g., CARTO® system sold by Biosense Webster) that is configured to obtain biometric data (e.g., anatomical and electrical measurements of a patient's organ, such as the heart 12 and as described herein) and perform a cardiac ablation procedure. More particularly, treatments for cardiac conditions such as cardiac arrhythmia often require obtaining a detailed mapping of cardiac tissue, chambers, veins, arteries and/or electrical pathways. For example, a prerequisite for performing a catheter ablation (as described herein) successfully is that the cause of the cardiac arrhythmia is accurately located in a chamber of the heart 12. Such locating may be done via an electrophysiological investigation during which electrical potentials are detected spatially resolved with a mapping catheter (e.g., the catheter 14) introduced into the chamber of the heart 12. This electrophysiological investigation, the so-called electro-anatomical mapping, thus provides 3D mapping data which can be displayed on the display device 27. In many cases, the mapping function and a treatment function (e.g., ablation) are provided by a single catheter or group of catheters such that the mapping catheter also operates as a treatment (e.g., ablation) catheter at the same time.

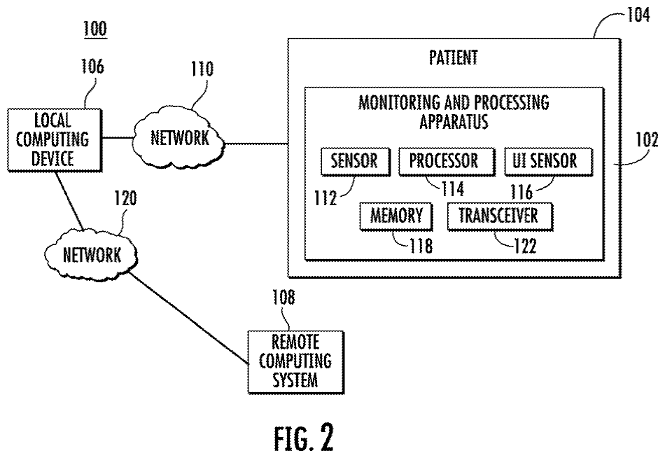

FIG. 2 is a block diagram of an example system 100 for remotely monitoring and communicating patient biometrics (i.e., patient data). In the example illustrated in FIG. 2, the system 100 includes a patient biometric monitoring and processing apparatus 102 associated with a patient 104, a local computing device 106, a remote computing system 108, a first network 110, a patient biometric sensor 112, a processor 114, a user input (UI) sensor 116, a memory 118, a second network 120, and a transmitter-receiver (i.e., transceiver) 122.

According to an embodiment, the patient biometric monitoring and processing apparatus 102 may be an apparatus that is internal to the patient's body (e.g., subcutaneously implantable), such as the catheter 14 of FIG. 1. The patient biometric monitoring and processing apparatus 102 may be inserted into a patient via any applicable manner including orally injecting, surgical insertion via a vein or artery, an endoscopic procedure, or a laparoscopic procedure.

According to an embodiment, the patient biometric monitoring and processing apparatus 102 may be an apparatus that is external to the patient, such as the electrode patches 38 of FIG. 1. For example, as described in more detail below, the patient biometric monitoring and processing apparatus 102 may include an attachable patch (e.g., that attaches to a patient's skin). The monitoring and processing apparatus 102 may also include a catheter with one or more electrodes, a probe, a blood pressure cuff, a weight scale, a bracelet or smart watch biometric tracker, a glucose monitor, a continuous positive airway pressure (CPAP) machine or virtually any device which may provide an input concerning the health or biometrics of the patient.

According to an embodiment, the patient biometric monitoring and processing apparatus 102 may include both components that are internal to the patient and components that are external to the patient.

The single patient biometric monitoring and processing apparatus 102 is shown in FIG. 2. Example systems may, however, may include a plurality of patient biometric monitoring and processing apparatuses. A patient biometric monitoring and processing apparatus may be in communication with one or more other patient biometric monitoring and processing apparatuses. Additionally or alternatively, a patient biometric monitoring and processing apparatus may be in communication with the network 110.

One or more patient biometric monitoring and processing apparatuses 102 may acquire patient biometric data (e.g., electrical signals, blood pressure, temperature, blood glucose level or other biometric data) and receive at least a portion of the patient biometric data representing the acquired patient biometrics and additional formation associated with acquired patient biometrics from one or more other patient biometric monitoring and processing apparatuses 102. The additional information may be, for example, diagnosis information and/or additional information obtained from an additional device such as a wearable device. Each of the patient biometric monitoring and processing apparatus 102 may process data, including its own acquired patient biometrics as well as data received from one or more other patient biometric monitoring and processing apparatuses 102.

Biometric data (e.g., patient biometrics, patient data, or patient biometric data) can include one or more of local activation times (LATs), electrical activity, topology, bipolar mapping, reference activity, ventricle activity, dominant frequency, impedance, or the like. The LAT can be a point in time of a threshold activity corresponding to a local activation, calculated based on a normalized initial starting point. Electrical activity can be any applicable electrical signals that can be measured based on one or more thresholds and can be sensed and/or augmented based on signal to noise ratios and/or other filters. A topology can correspond to the physical structure of a body part or a portion of a body part and can correspond to changes in the physical structure relative to different parts of the body part or relative to different body parts. A dominant frequency can be a frequency or a range of frequencies that are prevalent at a portion of a body part and can be different in different portions of the same body part. For example, the dominant frequency of a PV of a heart can be different than the dominant frequency of the right atrium of the same heart. Impedance can be the resistance measurement at a given area of a body part.

Examples of biometric data include, but are not limited to, patient identification data, intracardiac electrocardiogram (IC ECG) data, bipolar intracardiac reference signals, anatomical and electrical measurements, trajectory information, body surface (BS) ECG data, historical data, brain biometrics, blood pressure data, ultrasound signals, radio signals, audio signals, a two- or three-dimensional image data, blood glucose data, and temperature data. The biometrics data can be used, generally, to monitor, diagnosis, and treatment any number of various diseases, such as cardiovascular diseases (e.g., arrhythmias, cardiomyopathy, and coronary artery disease) and autoimmune diseases (e.g., type I and type II diabetes). Note that BS ECG data can include data and signals collected from electrodes on a surface of a patient, IC ECG data can include data and signals collected from electrodes within the patient, and ablation data can include data and signals collected from tissue that has been ablated. Further, BS ECG data, IC ECG data, and ablation data, along with catheter electrode position data, can be derived from one or more procedure recordings.

In FIG. 2, the network 110 is an example of a short-range network (e.g., local area network (LAN), or personal area network (PAN)). Information may be sent, via the network 110, between the patient biometric monitoring and processing apparatus 102 and the local computing device 106 using any one of various short-range wireless communication protocols, such as Bluetooth, Wi-Fi, Zigbee, Z-Wave, near field communications (NFC), ultraband, Zigbee, or infrared (IR).

The network 120 may be a wired network, a wireless network or include one or more wired and wireless networks. For example, the network 120 may be a long-range network (e.g., wide area network (WAN), the internet, or a cellular network,). Information may be sent, via the network 120 using any one of various long-range wireless communication protocols (e.g., TCP/IP, HTTP, 3G, 4G/LTE, or 5G/New Radio).

The patient biometric monitoring and processing apparatus 102 may include the patient biometric sensor 112, the processor 114, the UI sensor 116, the memory 118, and the transceiver 122. The patient biometric monitoring and processing apparatus 102 may continually or periodically monitor, store, process and communicate, via the network 110, any number of various patient biometrics. Examples of patient biometrics include electrical signals (e.g., ECG signals and brain biometrics), blood pressure data, blood glucose data and temperature data. The patient biometrics may be monitored and communicated for treatment across any number of various diseases, such as cardiovascular diseases (e.g., arrhythmias, cardiomyopathy, and coronary artery disease) and autoimmune diseases (e.g., type I and type II diabetes).

The patient biometric sensor 112 may include, for example, one or more sensors configured to sense a type of biometric patient biometrics. For example, the patient biometric sensor 112 may include an electrode configured to acquire electrical signals (e.g., heart signals, brain signals or other bioelectrical signals), a temperature sensor, a blood pressure sensor, a blood glucose sensor, a blood oxygen sensor, a pH sensor, an accelerometer and a microphone.

As described in more detail below, the patient biometric monitoring and processing apparatus 102 may be an ECG monitor for monitoring ECG signals of a heart (e.g., the heart 12). The patient biometric sensor 112 of the ECG monitor may include one or more electrodes for acquiring ECG signals. The ECG signals may be used for treatment of various cardiovascular diseases.

In another example, the patient biometric monitoring and processing apparatus 102 may be a continuous glucose monitor (CGM) for continuously monitoring blood glucose levels of a patient on a continual basis for treatment of various diseases, such as type I and type II diabetes. The CGM may include a subcutaneously disposed electrode, which may monitor blood glucose levels from interstitial fluid of the patient. The CGM may be, for example, a component of a closed-loop system in which the blood glucose data is sent to an insulin pump for calculated delivery of insulin without user intervention.

The transceiver 122 may include a separate transmitter and receiver. Alternatively, the transceiver 122 may include a transmitter and receiver integrated into a single device.

The processor 114 may be configured to store patient data, such as patient biometric data in the memory 118 acquired by the patient biometric sensor 112, and communicate the patient data, across the network 110, via a transmitter of the transceiver 122. Data from one or more other patient biometric monitoring and processing apparatus 102 may also be received by a receiver of the transceiver 122, as described in more detail below.

According to an embodiment, the patient biometric monitoring and processing apparatus 102 includes UI sensor 116 which may be, for example, a piezoelectric sensor or a capacitive sensor configured to receive a user input, such as a tapping or touching. For example, the UI sensor 116 may be controlled to implement a capacitive coupling, in response to tapping or touching a surface of the patient biometric monitoring and processing apparatus 102 by the patient 104. Gesture recognition may be implemented via any one of various capacitive types, such as resistive capacitive, surface capacitive, projected capacitive, surface acoustic wave, piezoelectric and infra-red touching. Capacitive sensors may be disposed at a small area or over a length of the surface such that the tapping or touching of the surface activates the monitoring device.

As described in more detail below, the processor 114 may be configured to respond selectively to different tapping patterns of the capacitive sensor (e.g., a single tap or a double tap), which may be the UI sensor 116, such that different tasks of the patch (e.g., acquisition, storing, or transmission of data) may be activated based on the detected pattern. In some embodiments, audible feedback may be given to the user from the patient biometric monitoring and processing apparatus 102 when a gesture is detected.

The local computing device 106 of the system 100 is in communication with the patient biometric monitoring and processing apparatus 102 and may be configured to act as a gateway to the remote computing system 108 through the second network 120. The local computing device 106 may be, for example, a, smart phone, smartwatch, tablet or other portable smart device configured to communicate with other devices via the network 120. Alternatively, the local computing device 106 may be a stationary or standalone device, such as a stationary base station including, for example, modem and/or router capability, a desktop or laptop computer using an executable program to communicate information between the patient biometric monitoring and processing apparatus 102 and the remote computing system 108 via the PC's radio module, or a USB dongle. Patient biometrics may be communicated between the local computing device 106 and the patient biometric monitoring and processing apparatus 102 using a short-range wireless technology standard (e.g., Bluetooth, Wi-Fi, ZigBee, Z-wave and other short-range wireless standards) via the short-range wireless network 110, such as a local area network (LAN) (e.g., a personal area network (PAN). In some embodiments, the local computing device 106 may also be configured to display the acquired patient electrical signals and information associated with the acquired patient electrical signals, as described in more detail below.

In some embodiments, the remote computing system 108 may be configured to receive at least one of the monitored patient biometrics and information associated with the monitored patient via network 120, which is a long-range network. For example, if the local computing device 106 is a mobile phone, network 120 may be a wireless cellular network, and information may be communicated between the local computing device 106 and the remote computing system 108 via a wireless technology standard, such as any of the wireless technologies mentioned above. As described in more detail below, the remote computing system 108 may be configured to provide (e.g., visually display and/or aurally provide) the at least one of the patient biometrics and the associated information to a healthcare professional (e.g., a physician).

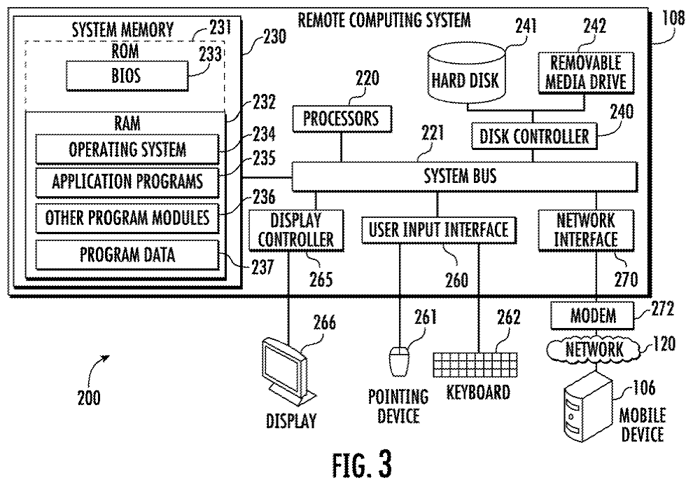

FIG. 3 is a system diagram of an example of a computing environment 200 in communication with network 120. In some instances, the computing environment 200 is incorporated in a public cloud computing platform (such as Amazon Web Services or Microsoft Azure), a hybrid cloud computing platform (such as HP Enterprise OneSphere) or a private cloud computing platform.

As shown in FIG. 3, computing environment 200 includes remote computing system 108 (hereinafter computer system), which is one example of a computing system upon which embodiments described herein may be implemented.

The remote computing system 108 may, via processors 220, which may include one or more processors, perform various functions. The functions may include analyzing monitored patient biometrics and the associated information and, according to physician-determined or algorithm driven thresholds and parameters, providing (e.g., via display 266) alerts, additional information or instructions. As described in more detail below, the remote computing system 108 may be used to provide (e.g., via display 266) healthcare personnel (e.g., a physician) with a dashboard of patient information, such that such information may enable healthcare personnel to identify and prioritize patients having more critical needs than others.

As shown in FIG. 3, the computer system 210 may include a communication mechanism such as a bus 221 or other communication mechanism for communicating information within the computer system 210. The computer system 210 further includes one or more processors 220 coupled with the bus 221 for processing the information. The processors 220 may include one or more CPUs, GPUs, or any other processor known in the art.

The computer system 210 also includes a system memory 230 coupled to the bus 221 for storing information and instructions to be executed by processors 220. The system memory 230 may include computer readable storage media in the form of volatile and/or nonvolatile memory, such as read only system memory (ROM) 231 and/or random-access memory (RAM) 232. The system memory RAM 232 may include other dynamic storage device(s) (e.g., dynamic RAM, static RAM, and synchronous DRAM). The system memory ROM 231 may include other static storage device(s) (e.g., programmable ROM, erasable PROM, and electrically erasable PROM). In addition, the system memory 230 may be used for storing temporary variables or other intermediate information during the execution of instructions by the processors 220. A basic input/output system 233 (BIOS) may contain routines to transfer information between elements within computer system 210, such as during start-up, that may be stored in system memory ROM 231. RAM 232 may comprise data and/or program modules that are immediately accessible to and/or presently being operated on by the processors 220. System memory 230 may additionally include, for example, operating system 234, application programs 235, other program modules 236 and program data 237.

The illustrated computer system 210 also includes a disk controller 240 coupled to the bus 221 to control one or more storage devices for storing information and instructions, such as a magnetic hard disk 241 and a removable media drive 242 (e.g., floppy disk drive, compact disc drive, tape drive, and/or solid-state drive). The storage devices may be added to the computer system 210 using an appropriate device interface (e.g., a small computer system interface (SCSI), integrated device electronics (IDE), Universal Serial Bus (USB), or FireWire).

The computer system 210 may also include a display controller 265 coupled to the bus 221 to control a monitor or display 266, such as a cathode ray tube (CRT) or liquid crystal display (LCD), for displaying information to a computer user. The illustrated computer system 210 includes a user input interface 260 and one or more input devices, such as a keyboard 262 and a pointing device 261, for interacting with a computer user and providing information to the processor 220. The pointing device 261, for example, may be a mouse, a trackball, or a pointing stick for communicating direction information and command selections to the processor 220 and for controlling cursor movement on the display 266. The display 266 may provide a touch screen interface that may allow input to supplement or replace the communication of direction information and command selections by the pointing device 261 and/or keyboard 262.

The computer system 210 may perform a portion or each of the functions and methods described herein in response to the processors 220 executing one or more sequences of one or more instructions contained in a memory, such as the system memory 230. Such instructions may be read into the system memory 230 from another computer readable medium, such as a hard disk 241 or a removable media drive 242. The hard disk 241 may contain one or more data stores and data files used by embodiments described herein. Data store contents and data files may be encrypted to improve security. The processors 220 may also be employed in a multi-processing arrangement to execute the one or more sequences of instructions contained in system memory 230. In alternative embodiments, hard-wired circuitry may be used in place of or in combination with software instructions. Thus, embodiments are not limited to any specific combination of hardware circuitry and software.

As stated above, the computer system 210 may include at least one computer readable medium or memory for holding instructions programmed according to embodiments described herein and for containing data structures, tables, records, or other data described herein. The term computer readable medium as used herein refers to any non-transitory, tangible medium that participates in providing instructions to the processor 220 for execution. A computer readable medium may take many forms including, but not limited to, non-volatile media, volatile media, and transmission media. Non-limiting examples of non-volatile media include optical disks, solid state drives, magnetic disks, and magneto-optical disks, such as hard disk 241 or removable media drive 242. Non-limiting examples of volatile media include dynamic memory, such as system memory 230. Non-limiting examples of transmission media include coaxial cables, copper wire, and fiber optics, including the wires that make up the bus 221. Transmission media may also take the form of acoustic or light waves, such as those generated during radio wave and infrared data communications.

The computing environment 200 may further include the computer system 210 operating in a networked environment using logical connections to local computing device 106 and one or more other devices, such as a personal computer (laptop or desktop), mobile devices (e.g., patient mobile devices), a server, a router, a network PC, a peer device or other common network node, and typically includes many or all of the elements described above relative to computer system 210. When used in a networking environment, computer system 210 may include modem 272 for establishing communications over a network 120, such as the Internet. Modem 272 may be connected to system bus 221 via network interface 270, or via another appropriate mechanism.

Network 120, as shown in FIGS. 2 and 3, may be any network or system generally known in the art, including the Internet, an intranet, a local area network (LAN), a wide area network (WAN), a metropolitan area network (MAN), a direct connection or series of connections, a cellular telephone network, or any other network or medium capable of facilitating communication between computer system 610 and other computers (e.g., local computing device 106).

Treatments for cardiac conditions such as cardiac arrhythmia often require obtaining a detailed mapping of cardiac tissue, chambers, veins, arteries and/or electrical pathways. For example, a prerequisite for performing a catheter ablation successfully is that the cause of the cardiac arrhythmia is accurately located in the heart chamber. Such locating may be done via an electrophysiological investigation during which electrical potentials are detected spatially resolved with a mapping catheter introduced into the heart chamber. This electrophysiological investigation, the so-called electro-anatomical mapping, thus provides 3D mapping data which can be displayed on a monitor. In many cases, the mapping function and a treatment function (e.g., ablation) are provided by a single catheter or group of catheters such that the mapping catheter also operates as a treatment (e.g., ablation) catheter at the same time.

Mapping of cardiac areas such as cardiac regions, tissue, veins, arteries and/or electrical pathways of the heart may result in identifying problem areas such as scar tissue, arrhythmia sources (e.g., electric rotors), healthy areas, and the like. Cardiac areas may be mapped such that a visual rendering of the mapped cardiac areas is provided using a display, as further disclosed herein. Additionally, cardiac mapping may include mapping based on one or more modalities such as, but not limited to local activation time (LAT), an electrical activity, a topology, a bipolar mapping, a dominant frequency, or an impedance. Data corresponding to multiple modalities may be captured using a catheter inserted into a patient's body and may be provided for rendering at the same time or at different times based on corresponding settings and/or preferences of a medial professional.

Cardiac mapping may be implemented using one or more techniques. As an example of a first technique, cardiac mapping may be implemented by sensing an electrical property of heart tissue, for example, local activation time, as a function of the precise location within the heart. The corresponding data may be acquired with one or more catheters that are advanced into the heart using catheters that have electrical and location sensors in their distal tips. As specific examples, location and electrical activity may be initially measured on about 10 to about 20 points on the interior surface of the heart. These data points may be generally sufficient to generate a preliminary reconstruction or map of the cardiac surface to a satisfactory quality. The preliminary map may be combined with data taken at additional points in order to generate a more comprehensive map of the heart's electrical activity. In clinical settings, it is not uncommon to accumulate data at 100 or more sites to generate a detailed, comprehensive map of heart chamber electrical activity. The generated detailed map may then serve as the basis for deciding on a therapeutic course of action, for example, tissue ablation, to alter the propagation of the heart's electrical activity and to restore normal heart rhythm.

Catheters containing position sensors may be used to determine the trajectory of points on the cardiac surface. These trajectories may be used to infer motion characteristics such as the contractility of the tissue. Maps depicting such motion characteristics may be constructed when the trajectory information is sampled at a sufficient number of points in the heart.

Electrical activity at a point in the heart may be typically measured by advancing a catheter containing an electrical sensor at or near its distal tip to that point in the heart, contacting the tissue with the sensor and acquiring data at that point. One drawback with mapping a cardiac chamber using a catheter containing only a single, distal tip electrode is the long period of time required to accumulate data on a point-by-point basis over the requisite number of points required for a detailed map of the chamber as a whole. Accordingly, multiple-electrode catheters have been developed to simultaneously measure electrical activity at multiple points in the heart chamber.

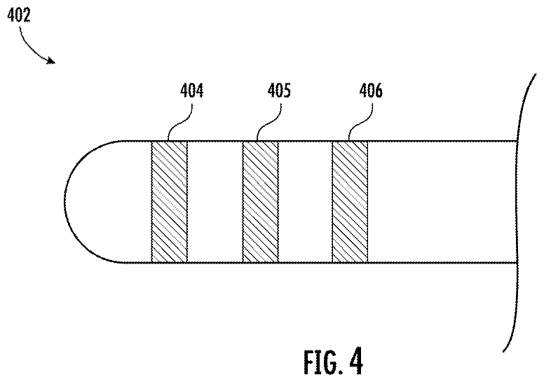

Multiple-electrode catheters may be implemented using any applicable shape such as a linear catheter with multiple electrodes, a balloon catheter including electrodes dispersed on multiple spines that shape the balloon, a lasso or loop catheter with multiple electrodes, or any other applicable shape. FIG. 4 shows an example of a linear catheter 402 including multiple electrodes 404, 405, and 406 that may be used to map a cardiac area. Linear catheter 402 may be fully or partially elastic such that it can twist, bend, and or otherwise change its shape based on received signal and/or based on application of an external force (e.g., cardiac tissue) on the linear catheter 402.

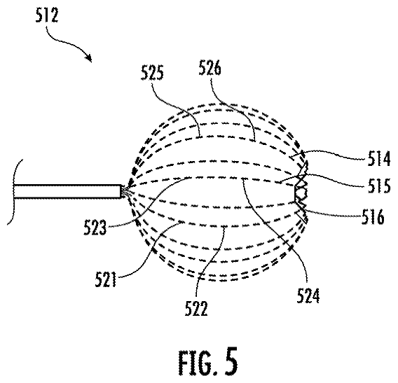

FIG. 5 shows an example of a balloon catheter 512 including multiple splines (e.g., 12 splines in the specific example of FIG. 5) including splines 514, 515, 516 and multiple electrodes on each spline including electrodes 521, 522, 523, 524, 525, and 526 as shown. The balloon catheter 512 may be designed such that when deployed into a patient's body, its electrodes may be held in intimate contact against an endocardial surface. As an example, a balloon catheter may be inserted into a lumen, such as a pulmonary vein (PV). The balloon catheter may be inserted into the PV in a deflated state such that the balloon catheter does not occupy its maximum volume while being inserted into the PV. The balloon catheter may expand while inside the PV such that electrodes on the balloon catheter are in contact with an entire circular section of the PV. Such contact with an entire circular section of the PV, or any other lumen, may enable efficient mapping and/or ablation.

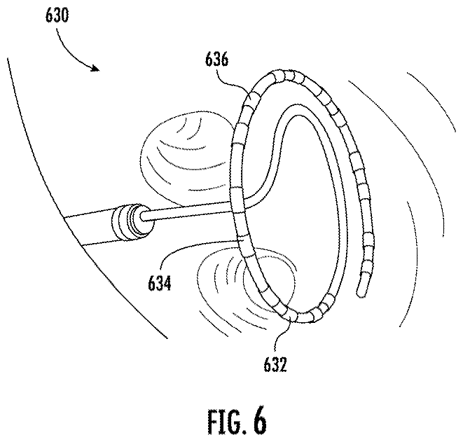

FIG. 6 shows an example of a loop catheter 630 (also referred to as a lasso catheter) including multiple electrodes 632, 634, and 636 that may be used to map a cardiac area. Loop catheter 630 may be fully or partially elastic such that it can twist, bend, and or otherwise change its shape based on received signal and/or based on application of an external force (e.g., cardiac tissue) on the loop catheter 630.

According to an example, a multi-electrode catheter may be advanced into a chamber of the heart. Anteroposterior (AP) and lateral fluorograms may be obtained to establish the position and orientation of each of the electrodes. Electrograms may be recorded from each of the electrodes in contact with a cardiac surface relative to a temporal reference such as the onset of the P-wave in sinus rhythm from a body surface ECG. The system, as further disclosed herein, may differentiate between those electrodes that register electrical activity and those that do not due to absence of close proximity to the endocardial wall. After initial electrograms are recorded, the catheter may be repositioned, and fluorograms and electrograms may be recorded again. An electrical map may then be constructed from iterations of the process above.

According to an example, cardiac mapping may be generated based on detection of intracardiac electrical potential fields. A non-contact technique to simultaneously acquire a large amount of cardiac electrical information may be implemented. For example, a catheter having a distal end portion may be provided with a series of sensor electrodes distributed over its surface and connected to insulated electrical conductors for connection to signal sensing and processing means. The size and shape of the end portion may be such that the electrodes are spaced substantially away from the wall of the cardiac chamber. Intracardiac potential fields may be detected during a single cardiac beat. According to an example, the sensor electrodes may be distributed on a series of circumferences lying in planes spaced from each other. These planes may be perpendicular to the major axis of the end portion of the catheter. At least two additional electrodes may be provided adjacent at the ends of the major axis of the end portion. As a more specific example, the catheter may include four circumferences with eight electrodes spaced equiangularly on each circumference. Accordingly, in this specific implementation, the catheter may include at least 34 electrodes (32 circumferential and 2 end electrodes).

According to another example, an electrophysiological cardiac mapping system and technique based on a non-contact and non-expanded multi-electrode catheter may be implemented. Electrograms may be obtained with catheters having multiple electrodes (e.g., between 42 to 122 electrodes). According to this implementation, knowledge of the relative geometry of the probe and the endocardium may be obtained such as by an independent imaging modality such as transesophageal echocardiography or 2D or 4D intracardiac echocardiogram (ICE). After the independent imaging, non-contact electrodes may be used to measure cardiac surface potentials and construct maps therefrom. This technique may include the following steps (after the independent imaging step): (a) measuring electrical potentials with a plurality of electrodes disposed on a probe positioned in the heart; (b) determining the geometric relationship of the probe surface and the endocardial surface; (c) generating a matrix of coefficients representing the geometric relationship of the probe surface and the endocardial surface; and (d) determining endocardial potentials based on the electrode potentials and the matrix of coefficients.

According to another example, a technique and apparatus for mapping the electrical potential distribution of a heart chamber may be implemented. An intra-cardiac multielectrode mapping catheter assembly may be inserted into a patient's heart. The mapping catheter assembly may include a multi-electrode array with an integral reference electrode, or, preferably, a companion reference catheter. The electrodes may be deployed in the form of a substantially spherical array. The electrode array may be spatially referenced to a point on the endocardial surface by the reference electrode or by the reference catheter which is brought into contact with the endocardial surface. The preferred electrode array catheter may carry a number of individual electrode sites (e.g., at least 24). Additionally, this example technique may be implemented with knowledge of the location of each of the electrode sites on the array, as well as a knowledge of the cardiac geometry. These locations are preferably determined by a technique of impedance plethysmography.

According to another example, a heart mapping catheter assembly may include an electrode array defining a number of electrode sites. The mapping catheter assembly may also include a lumen to accept a reference catheter having a distal tip electrode assembly which may be used to probe the heart wall. The mapping catheter may include a braid of insulated wires (e.g., having 24 to 64 wires in the braid), and each of the wires may be used to form electrode sites. The catheter may be readily positionable in a heart to be used to acquire electrical activity information from a first set of non-contact electrode sites and/or a second set of in-contact electrode sites.

According to another example, another catheter for mapping electrophysiological activity within the heart may be implemented. The catheter body may include a distal tip which is adapted for delivery of a stimulating pulse for pacing the heart or an ablative electrode for ablating tissue in contact with the tip. The catheter may further include at least one pair of orthogonal electrodes to generate a difference signal indicative of the local cardiac electrical activity adjacent the orthogonal electrodes.

According to another example, a process for measuring electrophysiologic data in a heart chamber may be implemented. The method may include, in part, positioning a set of active and passive electrodes into the heart, supplying current to the active electrodes, thereby generating an electric field in the heart chamber, and measuring the electric field at the passive electrode sites. The passive electrodes are contained in an array positioned on an inflatable balloon of a balloon catheter. In preferred embodiments, the array is said to have from 60 to 64 electrodes.

According to another example, cardiac mapping may be implemented using one or more ultrasound transducers. The ultrasound transducers may be inserted into a patient's heart and may collect a plurality of ultrasound slices (e.g., two dimensional or three-dimensional slices) at various locations and orientations within the heart. The location and orientation of a given ultrasound transducer may be known and the collected ultrasound slices may be stored such that they can be displayed at a later time. One or more ultrasound slices corresponding to the position of a probe (e.g., a treatment catheter) at the later time may be displayed and the probe may be overlaid onto the one or more ultrasound slices.

According to other examples, body patches and/or body surface electrodes may be positioned on or proximate to a patient's body. A catheter with one or more electrodes may be positioned within the patient's body (e.g., within the patient's heart) and the position of the catheter may be determined by a system based on signals transmitted and received between the one or more electrodes of the catheter and the body patches and/or body surface electrodes. Additionally, the catheter electrodes may sense biometric data (e.g., LAT values) from within the body of the patient (e.g., within the heart). The biometric data may be associated with the determined position of the catheter such that a rendering of the patient's body part (e.g., heart) may be displayed and may show the biometric data overlaid on a shape of the body part, as determined by the position of the catheter.

As noted above, a quality index may be defined as an outcome of a weight function that weighs several parameters of a data point, as listed, for example, in Table I below. Each candidate data point may be rated with a normalized score (also called smart index) ranging between 0 and 1.

An exemplary equation of data point smart index is:

$$\frac{{\sum }_{n = 1}^{N} parameter ’ s {weight}_{n} * parameter ’ s {score}_{n}}{{\sum }_{n = 1}^{N} parameter ’ s {weight}_{n}} , \qquad Eq . 1$$

where N is the total number of parameters (see Table I of parameters below), with each parameter given a weight, e.g., a score from 1 to 5. Again, the parameter's normalized score may be a number ranging between 0 and 1.

TABLE I
Parameters

| | Parameter | |
|---|---|---|
| Parameter Name | Weight | Parameter Score |
| Pattern matching | 5 | (−1) to 1 |
| | | According to the PM correlation score |
| CL | 2 | \|Current CL − median CL\| ≡ Δ |
| | | σ² = CL variance |
| | | $score = \{ \begin{matrix}1 - \frac{Δ}{{σ}^{2}} , & current CL is in range \\ 0 , & current CL is out range\end{matrix}$ |
| LAT stability | 3 | \|Current LAT − Prev. beat LAT\| ≡ Δ |
| | | $score = \{ \begin{matrix}1 - \frac{Δ}{12} , & Δ \leq 12 \\ 0 , & Δ > 12\end{matrix}$ |
| Complex data | 5 | 1/0 |
| point (e.g., LAM, | | |
| CFAE) | | |
| TPI | 4 | 1 (touch)/0 (no touch or unknown) |
| Position stability | 3 | \|Current position − Prev. beat position\| ≡ Δ |
| | | $score = \{ \begin{matrix}1 - \frac{Δ}{10} , & Δ \leq 10 \\ 0 , & Δ > 10\end{matrix}$ |
| Respiration | 5 | 1/0 (in/out respiration threshold) |
| Location (CPM) | 2 | 0.2 |
| SNR | 2 | $score = \{ \begin{matrix}\frac{S N R}{10} , & SNR \leq 10 \\ 1 , & SNR > 10\end{matrix}$ |
| \| dV/dt\| | 2 | $score = \{ \begin{matrix}\vert \frac{d V}{d t} \vert / 5 , & \vert \frac{d V}{d t} \vert \leq 5 \\ 1 , & \vert \frac{d V}{d t} \vert > 5\end{matrix}$ |

In Table I, complex fractionated atrial electrograms (CFAE) and late activation mapping (LAM) indicate complex EP signal behavior, such as those from a scarred tissue region. Because this deserves focus from the clinician, this distinction may therefore receive a high weight, at least for some arrhythmias.

In Table I, pattern matching is a test of data point consistency over cardiac cycles (a value of correlation between cardia cycles).
CL stands for cardia cycle length.
TPI stands for touch pressure index (or contact pressure index), which estimates contact force of a catheter electrode with a tissue wall during acquisition of the data point.

Position stability is a measure of electrode position consistency during acquisition over two or more cardiac cycles.

Respiratory weights adversely impact respiration on acquisition, such as introducing noise to signals.

Location CPM is a measure of the aforementioned ACL position tracking technique on electrode location consistency during acquisition.
SNR stands for the signal to noise ratio, which, if low, indicates a less robust acquisition.
   |dV/dt| reflects for the sharpness of the signal deflections.

Table I is provided by way of example only. Other or different parameter lists may be utilized.

A quality profile of the points acquired, or attempted to be acquired, for the mapping from the system of FIG. 1, may be defined. This quality profile may be provided to a user on the screen 20, for example, of FIG. 1. The quality profile may be defined below. The quality profile provides for additional information beyond the single dimension quality index or smart index. As the current quality index provides a single point of feedback for each data point collected, many data points currently are rejected when the quality index registers low because of contributing factors that may be less concerning to an operator driving the low value, and alternatively, many data points are accepted when the quality index registers high because of contributing factors that may be less defining to an operator driving the high value. That is, a single value of quality index can result in data points having the same quality index for different reasons and being driven to the quality index with one or more dominant parameters that lead to the final quality index. This dominant parameter may be unimportant (or less important) generally but dominating the point being accepted/rejected. The below quality profile provides feedback to a user using additional values to allow the user to understand the underlying quality of the data point.

A system, device and method for registering a mapped data point is described. The system, device and method may include a catheter, a processor and memory. The system, device and method include providing a plurality of values in a quality profile of a mapped data point of an electro-anatomical map, and displaying, via an interface, a simplex having vertices defined by each of the plurality of values. The system, device and method may include acquiring a data point if each of the plurality of values are within the defined range for a mapped data point the data point. The simplex may be a triangle and the plurality of values are composed of a first value, a second value, and a third value. The first value may include morphology quality, the second value may include annotation quality, and the third value may include location quality. The simplex may be a tetrahedron and the plurality of values are composed of a first value, a second value, a third value, and a fourth value. The plurality of values may include underlying parameters for measured values. The one of the plurality of values that includes underlying parameters for measured values may combine the underlying parameters to determine the one value using weighting and the weighting may be equal between parameters or the weighting may include a dominant parameter(s) and other complementary parameters. The plurality of values comprises a value for pattern matching of the data point. The plurality of values comprises a value for LAT stability of the data point and a value for sharpness of the data point, and the plurality of values comprises a value for positional stability, a TPI value, a CPM value and a value for respiration. The system, device and method may further include displaying, via the interface, a visual depiction of a defined range on the simplex, the defined range including of acceptable values for the plurality of values in the quality profile.

The system, method and device may be defined using a barycentric coordinate system, which is a coordinate system in which the location of a point is specified by reference to a simplex, such as a triangle for points in a plane, a tetrahedron for points in three-dimensional space, etc., for example The barycentric coordinates of a point may be interpreted as masses placed at the vertices of the simplex, such that the point that is the center of mass (or barycenter) of these masses. The simplex represents a generalization of the notion of a triangle or tetrahedron in arbitrary dimensions. For example, a 0-dimensional simplex is a point, a 1-dimensional simplex is a line segment, a 2-dimensional simplex is a triangle, a 3-dimensional simplex is a tetrahedron, and a 4-dimensional simplex is a 5-cell. As described below, the description may focus on particular dimensioned figure, for ease of depiction, description and understanding, while it is understood that each dimensioned figure represents a simplex in arbitrary dimensions defined dimensionally by the number of variables being depicted.

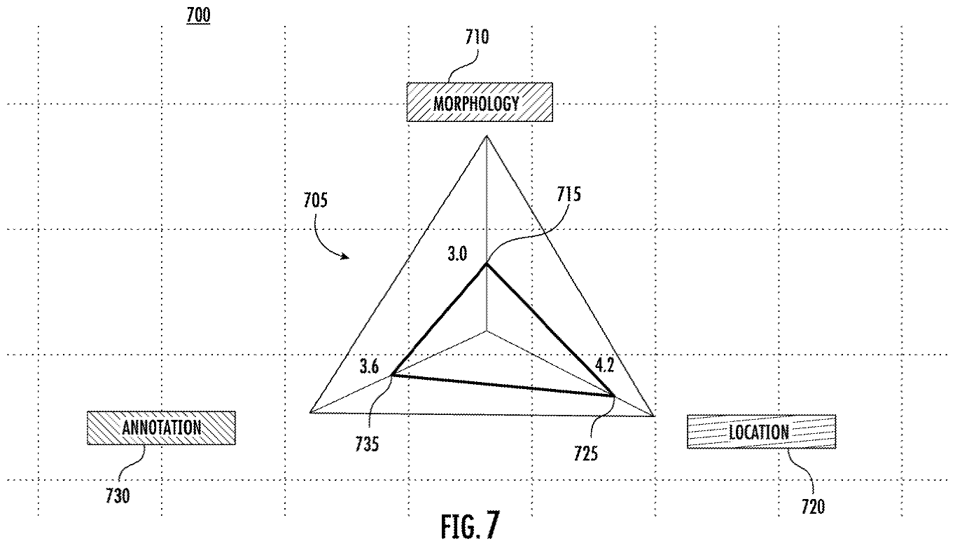

FIG. 7 illustrates a quality profile 700 of a CARTO point. Each point's quality is assessed by calculating a score for a plurality of parameters, such as the parameters of Table I. These parameters may include, or may be grouped into, metrics such as, but not limited to, annotation quality 730, morphology quality 710 and location quality 720. Other example parameters include cycle length, signal amplitude, stability of local activation time (LAT), electrode stability and level of contact touch with tissue, as more fully described above. Each parameter may be scored on a scoring scale. The scale may be from 0-5, 0-10, 0-100, for example, for each parameter. The respective parameters may each be made up of underlying parameters that factor into the specific parameter score. More details on the underlying parameters are set forth below. The CARTO system relies on the use of multiple parameters to assess point quality. These parameters may be assigned into one of these three parameters groups: Morphology, Annotation, Location.

The quality profile 700 is represented a simplex using a shape where the values define the vertices. For example, quality profile 700 may be represented with a simplex, such as a line with two values, a triangle with three values, a tetrahedron with four values, etc.

As would be understood by those possessing an ordinary skill in the pertinent arts, additional parameters may be added. These additional parameters may be assigned to the parameters groups described herein. Additional parameter groups may be designed to classify and handle a new quality parameter that does not fit into one of these discussed parameters groups. As described, with the parameter groups, the interface is simplified and captures the most important parameters.

Specifically, in the example of FIG. 7, the morphology quality 710 is illustrated at point 715 as a 3.0 out of 5. The location quality 720 is illustrated at point 725 as a 4.2 out of 5. The annotation quality 730 is illustrated at point 735 as a 3.6 out of 5. The quality profile 700 is represented using a simplex with three points, in this case, by the triangle defined by point 715, point 725 and point 735 represented as simplex 705. This simplex 705 provides more detailed information to a user regarding the actual quality of a given data point.

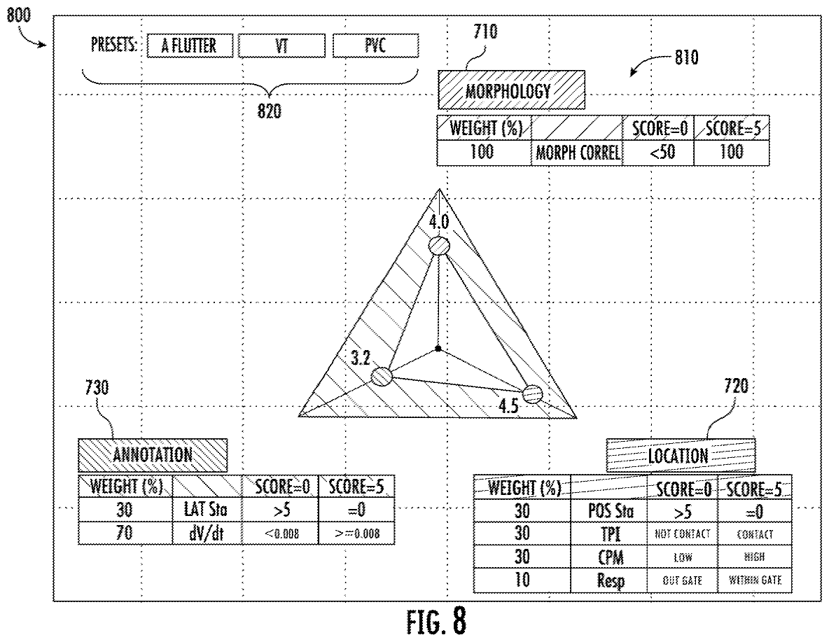

FIG. 8 illustrates a screenshot 800 related to display 20 including a quality profile 810. Quality profile 810 is similar to quality profile 700 of FIG. 7. Screenshot 800 includes quality profile 810 as well as template setting 820. Template setting 820 permits pre-defined setting for flutter, VT and PVC as described above. Since different arrhythmia may have different quality profiles needed or desired for mapping, using templates defined by template setting 820 may help the user to easily set the system for the appropriate profile based on the patient arrhythmia.

Quality profile 810 includes annotation quality 730, morphology quality 710 and location quality 720 as described above with respect to quality profile 700. Within quality profile 810, morphology quality 710 is comprised of morphology correlation that includes pattern matching. As illustrated in FIG. 8, a morphology correlation of less than 50 leads to a morphology quality 710 value of 0. A morphology correlation of 100 leads to a morphology quality 710 value of 5. Morphology correlation is weighted at 100% of the input for the morphology quality 710. The value of morphology quality 710 between 0 and 5 may be configured in a linear fashion based on the morphology correlation between 50 and 100, for example. Alternatively, a second order effect may be used as the morphology correlation registers from 50 to 100. Using a linear scaling, since 50 leads to 0 and 100 leads to 5, a correlation of 75 results in a score of 2.5.

The value of the pattern matching is the input for the value of morphology correlation. While other elements may also be used in determining morphology quality 710, in the illustrated example quality profile 810, the morphology correlation is the only element within the morphology quality 710. Pattern matching is the designation for the correlation between a baseline template pattern acquired by the user and the pattern of the current beat. If the current beat matches exactly the baseline template pattern the score of the matching is 1.0. The lower the score the less resemblance between the baseline pattern and the current pattern. The pattern matching morphology correlation is a correlation score between 0-100%. Where 100% means an identical morphology between the baseline template and the current beat. In the present example, any score of pattern matching morphology correlation below 50 would provide a 0 Morphology score in the quality profile.

The morphology quality 710 illustrated in FIG. 8 is 4.0 and denoted with a mark within display 800 using the simplex described above.

Within quality profile 700, location quality 720 includes position stability, TPI, CPM and respiration. Location quality 720 may include the position stability, TPI, CPM and respiration with equal weighting contributing to the value of location quality 720. Alternatively, or additionally, these elements of location quality 720 may be weighted by a user in any defined manner. Alternatively, or additionally, location quality 720 may include the elements with a dominate factor and the other elements contributing to the value for location quality 720 in a slight, or smaller, manner.

Position stability (POS stability) is the designation to represent the difference in location stability between consecutive beats. Generally, position stability of 0 means that there is no difference between the previous beat location and the current beat location. That is, that the position of the point is stable. The POS stability, generally, may be in a range between 0-10 mm.

TPI (Tissue Proximity Indicator) is the designation of an indicator to the user whether the electrode is in contact or not in contact with the tissue based on impedance technology. In the CARTO interface, there are generally three values for TPI. TPI is assessed for each electrode as being in contact, not in contact, or unknown.

CPM (Current Position Mapping) is the designation that is also referred to as a Visualization Matrix. The quality of the visualization matrix at a specific place can affect the quality of the position of a point acquired within it. Generally, CPM may have the following values: 0—no CPM Matrix, 1—Red-Low Quality CPM Matrix, 2—Blue-Intermediate Quality CPM Matrix, or 3—Green-High Quality CPM Matrix.

Respiratory includes respiration gating that when activated a point can be acquired only if it is within the respiratory gating threshold. Usually, the respiratory gating threshold is set in such a way that the point is acquired in the experium phase of the respiration cycle. Generally, respiratory may be assigned one of two values often referred to as taken out of the gate and taken within the gate referring to the experium phase.

In the example provided in FIG. 8, location quality 720 may include the POS stability with a weighting of 30%, TPI with a weighting of 30%, CPM with a weighting of 30% and respiration with a weighting of 10% contributing to the value of location quality 720. The four parameters are combined using the weighting 30, 30, 30, 10 set forth above for the parameters to provide the contribution of each to the value of location quality 720.

With respect to POS stability and its 30% contribution to location quality 720, a POS stability score of greater than 5 provides a location quality score contribution of 0. When the POS stability score is 0 it provides a location quality score contribution of 5. The contribution of POS stability to the location quality score may be linear in between 0 and 5 for scores between 5 and 0, for example. Using a linear scaling, since a POS stability score of greater than 5 provides a location quality score contribution of 0 and the POS stability score is 0 it provides a location quality score contribution of 5, a POS stability of 2.5 provides a location quality score contribution of 2.5.

With respect to TPI and its 30% contribution to location quality 720, a TPI score of greater than 1 provides a location quality score contribution of 0. In FIG. 8, a score of 5 is assigned to points “in contact,” a score of 0 is assigned to points that are “not in contact” or “unknown” TPI is an example of a binary parameter. As a result, TPI is assigned a quality score of either 0 or 5.

With respect to CPM and its 30% contribution to location quality 720, since CPM can have the following values: 0—no CPM Matrix, 1—Red-Low Quality CPM Matrix, 2—Blue-Intermediate Quality CPM Matrix, or 3—Green-High Quality CPM Matrix, a zone 3 high quality CPM provides a location quality score contribution of 5, a zone 1 low quality CPM provides a location quality score of 0, and zone 2 intermediate quality CPM provides a location quality score of 2.5

With respect to respiration and its 10% contribution to location quality 720, since respiratory may be assigned one of two value often referred to as taken out of the gate and taken within the gate referring to the experium phase, a location quality score contribution of 0 may be assigned to any point that was taken out of the gate, and a location quality score contribution of 5 may be assigned to any point that was taken within the gate.

The POS stability value, TPI, CPM and respiration values may be combined using the weighting to determine the overall location quality 720. As illustrated in FIG. 8, the location quality 720 is 4.5 and denoted with a mark within display 800 using the simplex described above.

Within quality profile 810, annotation quality 730 includes LAT stability and sharpness (dv/dt), for example. Annotation quality 730 may include the LAT stability and sharpness with equal weighting contributing to the value of annotation quality 730. Alternatively, or additionally, these elements of annotation quality 730 may be weighted by a user in any defined manner. Alternatively, or additionally, annotation quality 730 may include the elements with a dominate factor and the other element(s) contributing to the value for annotation quality 730 in a slight manner. As illustrated in FIG. 8, annotation quality 730 may include the LAT stability and sharpness with the LAT stability value being weighted 30% and the sharpness value weighted 70%.

LAT stability is the designation for a value that represents the difference between the LAT of consecutive beats. A LAT stability of 0 means that there is no difference between the previous beat and the current beat. That is, that the LAT is stable. The LAT stability value range is usually between 0-12 ms.

Sharpness (dV/dt) is a designation to represent the sharpness of a signal, which is the first derivative of the voltage. The steeper the signal the higher the dV/dt. Generally, bipolar signals have a dV/dt threshold value of approximately 0.008 mV/ms. A dV/dt value greater than 0.008 mV/ms provides a sharp signal. A dV/dt value less than or equal to 0.008 mV/ms provides a shallow signal. As illustrated in FIG. 8, a dV/dt value of 0.008 mV/ms and above is assigned a score of 5. A dV/dt value of less than 0.008 mV/ms is assigned a score of 0.

With respect to LAT stability and its 30% contribution to annotation quality 730, a LAT stability score of greater than 5 provides an annotation quality score contribution of 0. When the LAT stability score is 0 it provides an annotation quality score contribution of 5. The contribution of LAT stability to the annotation quality score may be linear in between 0 and 5 for scores between 5 and 0, for example. Using a linear scaling, since greater than 5 leads to 0 and 0 leads to 5, a LAT stability score of 2.5 provides an annotation quality score contribution of 2.5.

With respect to sharpness and its 70% contribution to annotation quality 730, a sharpness score of less than 2 provides an annotation quality score contribution of 0. When the sharpness score is greater than or equal to 12, it provides an annotation quality score contribution of 5. The contribution of sharpness to the annotation quality score may be linear in between 0 and 5 for scores between 2 and 12, for example. Using a linear scaling, since less than 2 leads to 0 and greater than or equal to 12 leads to 5, a sharpness core of 7 provides an annotation quality score contribution of 2.5.

The LAT stability and sharpness values may be combined using the weighting to determine the overall annotation quality 730. As illustrated in FIG. 8, the annotation quality 730 is 3.2 and denoted with a mark within display 800 using the simplex described above.

While the above discussion generally focuses on a linear scaling between the exemplary ranges and scores discussed, other types of fitting may be used to extrapolate between points, including for example, fitting data using a nonlinear form such as a quadratic equation, or some other fitting that is understood by those possessing an ordinary skill in the art. This other fitting may be applied to any of the liner scaling examples described herein.

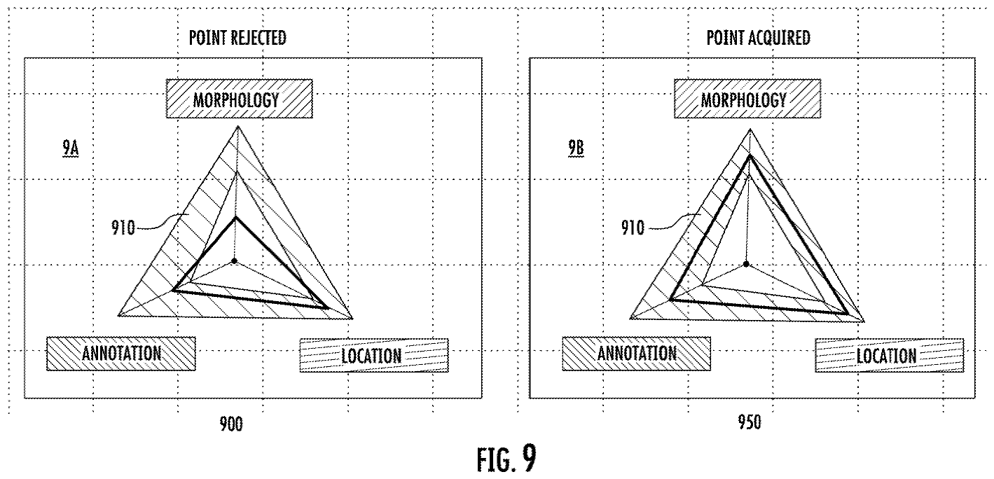

FIG. 9 illustrates two example quality profiles 9A 900, 9B 950 illustrating the use of the quality profile in real-time to accumulate data. In example 9A 900, there is a representation of a point being taken where the quality profile is compared using the three parameters of annotation quality 730, morphology quality 710 and location quality 720 as described herein. In example 9A 900, the annotation quality 730 and location quality 720 are provided as shown by each registering within the simplex. The morphology quality 710, however, is outside the acceptable limits (the point simplex is drawn inside the acceptable simplex) of the defined range 910. As such, the point represented in example 9A 900 is rejected.

In example 9B 950, there is a representation of a point being taken where the quality profile is compared using the three parameters of annotation quality 730, morphology quality 710 and location quality 720 as described herein. In example 9B 950, annotation quality 730, morphology quality 710 and location quality 720 are provided as shown by each registering within the simplex. Since annotation quality 730, morphology quality 710 and location quality 720 are each within the defined range 910, the point represented in the example 9B 950 is within acceptable limits. As such, the point represented in example 9B 950 is acquired.

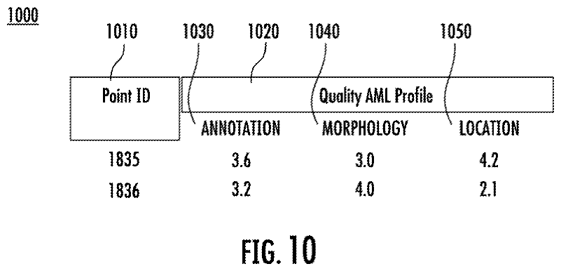

FIG. 10 illustrates a point list 1000 which may be created by acquiring and recording the points in a mapping. A point list is one of the features in the CARTO system. As illustrated in FIG. 10, the quality parameters are additional fields that can be added to the current point list. The point list 1000 includes the point ID 1010 of each point along with the associated quality profile 1020 as described herein. The quality profile 1020 may include the parameter scores for the annotation quality 1030, morphology profile 1040 and location profile 1050 registered for each point. For example, as illustrated a point with a point ID 1010 of 1835 is listed with a quality profile 1020 having an annotation profile 1030 of 3.6, a morphology profile 1040 of 3.0 and a location profile 1050 of 4.2. As would be understood, this point may be displayed on the simplex described herein. For example, as illustrated a point with a point ID 1010 of 1836 is listed with a quality profile 1020 having an annotation profile 1030 of 3.2, a morphology profile 1040 of 4.0 and a location profile 1050 of 2.1. As would be understood this point may be displayed on the simplex described herein. The point list 1000, although not shown in FIG. 10, may include all the other points recorded and acquired in a mapping.

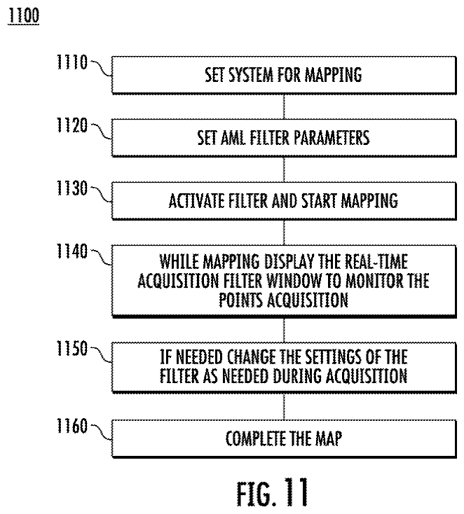

FIG. 11 illustrates a method 1100 of using the quality profile. Method 1100 includes setting the system for mapping at 1110. At 1120, method 1100 includes setting the AML filter parameters. As described herein, the setting of the AML filter parameters at 1120 may include defining the specific filter values that are relevant to a specific arrhythmia, for example, and may also include predefined preset values of the filter parameters. The AML filter parameters are the parameters that are described in FIG. 8.

At 1130, method 1100 includes activating the filter and starting mapping. At 1140, method 1100 includes monitoring the points being acquired while mapping displays the real-time acquisition filter window. This monitoring at 1140 is described above with respect to FIG. 9A and FIG. 9B, for example.

At 1150, method 1100 includes, as needed, changing the filter settings during the acquisition. Such changing at 1150 may be based on acquiring too many or too few points, rejecting quality points, or accepting non-quality points, for example. At 1160, method 1100 includes completing the mapping.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. Examples of computer-readable media include electronic signals (transmitted over wired or wireless connections) and computer-readable storage media. Examples of computer-readable storage media include, but are not limited to, a read only memory (ROM), a random-access memory (RAM), a register, cache memory, semiconductor memory devices, magnetic media such as internal hard disks and removable disks, magneto-optical media, and optical media such as CD-ROM disks, and digital versatile disks (DVDs).

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. A computer readable medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Examples of computer-readable media include electrical signals (transmitted over wired or wireless connections) and computer-readable storage media. Examples of computer-readable storage media include, but are not limited to, a register, cache memory, semiconductor memory devices, magnetic media such as internal hard disks and removable disks, magneto-optical media, optical media such as compact disks (CD) and digital versatile disks (DVDs), a random-access memory (RAM), a read-only memory (ROM), an erasable programmable read-only memory (EPROM or Flash memory), a static random-access memory (SRAM), and a memory stick. A processor in association with software may be used to implement a radio frequency transceiver for use in a terminal, base station, or any host computer.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one more other features, integers, steps, operations, element components, and/or groups thereof.

The descriptions of the various embodiments herein have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer implemented method comprising:
providing a plurality of values in a quality profile of a mapped data point of an electro-anatomical map; and
displaying, via an interface, a simplex having vertices defined by each of the plurality of values.

2. The method of claim 1, wherein if each of the plurality of values are within the defined range for a mapped data point the data point is acquired.

3. The method of claim 1, wherein the simplex is a triangle and the plurality of values are composed of a first value, a second value, and a third value.

4. The method of claim 3, wherein the first value comprises morphology quality, the second value comprises annotation quality, and the third value comprises location quality.

5. The method of claim 1, wherein the simplex is a tetrahedron and the plurality of values are composed of a first value, a second value, a third value, and a fourth value.

6. The method of claim 1, wherein the plurality of values includes underlying parameters for measured values.

7. The method of claim 6, wherein the one of the plurality of values that includes underlying parameters for measured values combines the underlying parameters to determine the one value using weighting.

8. The method of claim 7, wherein the weighting is equal between parameters.

9. The method of claim 7, wherein the weighting includes a dominant parameters and other complementary parameters.

10. The method of claim 1, wherein the plurality of values comprises a value for pattern matching of the data point.

11. The method of claim 1, wherein the plurality of values comprises a value for LAT stability of the data point and a value for sharpness of the data point.

12. The method of claim 1, wherein the plurality of values comprises a value for positional stability, a TPI value, a CPM value and a value for respiration.

13. The method of claim 1 further comprising displaying, via the interface, a visual depiction of a defined range on the simplex, the defined range including of acceptable values for the plurality of values in the quality profile.

14. A system for registering a mapped data point comprising:
a processor; and
a memory communicatively coupled to the processor,
the processor and memory operating to:
provide a plurality of values in a quality profile of a mapped data point of an electro-anatomical map; and
display, via an interface, a simplex having vertices defined by each of the plurality of values.

15. The system of claim 14, wherein if each of the plurality of values are within the defined range for a mapped data point the data point is acquired.

16. The system of claim 14, wherein the plurality of values includes underlying parameters for measured values.

17. The system of claim 14, wherein the simplex is a triangle and the plurality of values are composed of a first value, a second value, and a third value.

18. The system of claim 17, wherein the first value comprises morphology quality, the second value comprises annotation quality, and the third value comprises location quality.

19. The system of claim 14, wherein the plurality of values comprises at least one of a value for pattern matching of the data point, a value for LAT stability of the data point and a value for sharpness of the data point, and a value for positional stability, a TPI value, a CPM value and a value for respiration.

20. The system of claim 14, wherein the processor and memory further operate to display, via the interface, a visual depiction of a defined range on the simplex, the defined range including of acceptable values for the plurality of values in the quality profile.
